(19) United States
(12) Patent Application Publication
(10) Pub. No.: US 2024/0010984 A1
(43) Pub. Date: Jan. 11, 2024

(54) SERUM-FREE MEDIUM FOR DIFFERENTIATION OF A PROGENITOR CELL

(71) Applicant: Mosa Meat B.V., Maastricht (NL)

(72) Inventors: Helder CRUZ, Maastricht (NL); Joshua Edwin FLACK, Maastricht (NL); Carolina FURQUIM, Maastricht (NL); Iva KLEVERNIC, Maastricht (NL); Lea MELZENER, Maastricht (NL); Tobias MEßMER, Maastricht (NL); Mark POST, Maastricht (NL); Anon VAN ESSEN, Maastricht (NL)

(21) Appl. No.: 18/254,264

(22) PCT Filed: Nov. 25, 2021
(86) PCT No.: PCT/NL2021/050718

§ 371 Date: May 24, 2023

(30) Foreign Application Priority Data
Nov. 25, 2020 (NL) 2026978

(51) Int. Cl. C12N 5/077; A23L 13/00

(52) U.S. Cl. CPC C12N 5/0658; A23L 13/00; C12N 2500/90; C12N 2501/11; C12N 2501/42; C12N 2501/335; C12N 2501/80; C12N 2500/42; C12N 2500/24; C12N 2500/34; C12N 2500/38; C12N 2500/46

(57) ABSTRACT

The invention relates inter alia to a method for differentiating a muscle progenitor cell, comprising the step of: —culturing a muscle progenitor cell in a serum-free medium for differentiating a muscle progenitor cell, wherein said serum-free medium comprises —at least one differentiation inducer selected from the group consisting of a lysophosphatidic acid receptor 1 (LPAR1) agonist, a lysophosphatidic acid receptor 3 (LPAR3) agonist, an oxytocin receptor (OXTR) agonist, a glucagon receptor (GCGR) agonist, a lactate and a Notch signaling pathway inhibitor.

### FIELD OF THE INVENTION

The invention is in the field of serum-free media for animal cell culture, more specifically a serum-free medium for use in methods of differentiating bovine progenitor cells such as bovine muscle progenitor cells, into myocytes. The invention also relates to methods for differentiating such progenitor cells by cell culture using a culture medium as disclosed herein, and meat products obtained with such methods. Prior to differentiation, said progenitor cells may have been subjected to proliferation in a serum-free medium e.g. as part of an entirely serum-free cell culture process for the production of cultured meat for human consumption.

### BACKGROUND TO THE INVENTION

Serum from varied origins has been used as an essential component in animal cell culture media, since it provides several important nutrients, vitamins, growth factors and adhesion proteins, among other components.

However, serum is a potential source of contaminants such as bacteria, mycoplasma, viruses, and prions, with the latter being a more recent source of concern since they are agents of transmissible neurodegenerative diseases in humans and other animals, from which serum is most often obtained, such as from bovines. These considerations play an important role, especially when the animal cell types are cultured for food applications, such as cell culture-based meat production for human consumption.

Moreover, serum represents a high cost to the bioprocesses and introduces variability in performance given the lot to lot variation of sera. In addition, the animal well-being is a source of concern if serum is used in cell culture applications.

Myogenic differentiation of progenitor cells obtained from different tissue sources, often muscle tissue, is traditionally performed by decreasing the percentage of serum in the culture medium, often decreasing serum from 20% (v/v) into 2% (v/v). A myogenic differentiation medium, that does not contain serum, and which induces differentiation of progenitor cells that are already proliferating and/or expanding under serum-free culture conditions, has not been developed before. This is especially relevant if the serum-free differentiation medium is used to differentiate progenitor cells which have been proliferated previously in serum-free proliferation medium, in an entirely serum-free cell culture process, in which conditions inducing cell differentiation by the established reference method of reducing serum concentration is not possible.

There is thus a need for a serum-free cell culture medium that allows for differentiation of progenitor cells, for instance when such cells have previously been proliferated (expanded) under serum-free conditions. There is especially a need for such a medium in cell culture-based meat production for human consumption.

### SUMMARY OF THE INVENTION

The present inventors discovered a serum-free medium that can be used for differentiation of non-human, mammalian progenitor cells such as bovine, ovine and porcine progenitor cells, preferably bovine, ovine or porcine muscle progenitor cells.

Therefore, the invention provides in one aspect a method for differentiating a muscle progenitor cell, comprising the step of:—culturing a muscle progenitor cell in a serum-free medium for differentiating a muscle progenitor cell, wherein said serum-free medium comprises—at least one differentiation inducer selected from the group consisting of a lysophosphatidic acid receptor 1 (LPAR1) agonist, a lysophosphatidic acid receptor 3 (LPAR3) agonist, an oxytocin receptor (OXTR) agonist, a glucagon receptor (GCGR) agonist, a lactate and a Notch signaling pathway inhibitor.

From RNA sequencing data, it was surprisingly established that certain cell surface receptors of muscle-tissue derived progenitor cells are upregulated during the process of differentiation of said progenitor cells into myocytes (Example 1). By incorporating at least one agonist (inducer) of the identified overexpressed receptors (i.e. at least one of an agonist of LPAR1, LPAR3, OXTR or GCGR) or lactate into a serum-free culture medium, it was surprisingly found that myogenic differentiation could in fact be induced (Example 2). The data also show that it is possible to achieve differentiation by adding one or more of the receptor agonists in a range of concentrations (Example 3). Further, it was discovered that in a subpopulation of bovine muscle progenitor cells Notch2 and Notch3 receptors are upregulated (Example 4), and that inhibiting Notch pathway signaling further improves myogenic differentiation (Example 5; FIG. 3).

Another important and unexpected achievement of the inventors was that they were able to define serum-free culture media that can be used for differentiation of muscle progenitor cells, especially bovine, ovine or porcine muscle progenitor cells, such a serum-free culture medium containing one or more of the above-mentioned differentiation inducers.

Another important and unexpected achievement of the inventors was that they were able to define a serum-free culture medium that can be used for differentiation of progenitor cells which have been proliferated previously in a serum-free proliferation medium, in an entirely serum-free cell culture process. In an entirely serum-free cell culture process, induction of cell differentiation by the established and widely used reference method of reducing serum concentration is not possible.

Advantages of a serum-free medium of the invention are that it does not contain serum and is preferably also animal component-free. Such a serum-free culture medium can be produced at significantly lower costs than culture media that contain serum, which is a requirement in order to obtain a viable cell culture-based meat product. In addition, the use of such a serum-free culture medium in cell culture applications satisfies current food regulations, which is beneficial when developing a cell culture-based meat product as disclosed herein.

In a preferred embodiment of a method for differentiating of the invention, said muscle progenitor cell is a proliferated muscle progenitor cell (i.e. is a muscle progenitor cell that has been proliferated or expanded in cell culture after isolation from a suitable tissue source). In other words, in a preferred embodiment, said muscle progenitor cell is a muscle progenitor cell that originates from, or is derived of, a cell culture of proliferating or expanding muscle progenitor cells, preferably wherein said cell culture is a cell culture that is comprised in a serum-free (proliferation or expansion) medium.

In another preferred embodiment of method for differentiating of the invention, said serum-free medium comprises (i) at least one differentiation inducer selected from the group consisting of a lysophosphatidic acid receptor 1 (LPAR1) agonist, a lysophosphatidic acid receptor 3 (LPAR3) agonist, an oxytocin receptor (OXTR) agonist, a glucagon receptor (GCGR) agonist and a lactate, and (ii) a Notch signaling pathway inhibitor. In embodiments, said serum-free medium comprises (i) a lysophosphatidic acid receptor 1 (LPAR1) agonist and a Notch signaling pathway inhibitor, (ii) a lysophosphatidic acid receptor 3 (LPAR3) agonist and a Notch signaling pathway inhibitor, (iii) an oxytocin receptor (OXTR) agonist and a Notch signaling pathway inhibitor, (iv) a glucagon receptor (GCGR) agonist and a Notch signaling pathway inhibitor or (v) a lactate and a Notch signaling pathway inhibitor.

In another preferred embodiment of a method for differentiating of the invention, said lysophosphatidic acid receptor 1 (LPAR1) agonist and/or said lysophosphatidic acid receptor 3 (LPAR3) agonist is a lysophosphatidic acid.

In another preferred embodiment of a method for differentiating of the invention, said oxytocin receptor (OXTR) agonist is oxytocin.

In another preferred embodiment of a method for differentiating of the invention, said glucagon receptor (GCGR) agonist is glucagon.

In another preferred embodiment of a method for differentiating of the invention, said Notch signaling pathway inhibitor is a gamma-secretase inhibitor.

In another preferred embodiment of a method for differentiating of the invention, said Notch signaling pathway inhibitor is a compound selected from the group consisting of DAPT, E2012, L685458, R04929097 and LY-411575, preferably DAPT.

In another preferred embodiment of a method for differentiating of the invention, said method is a method for proliferating a muscle progenitor cell followed by differentiating proliferated muscle progenitor cells, wherein said method further comprises, prior to differentiating said muscle progenitor cell, a step of:—culturing a muscle progenitor cell in a serum-free medium for proliferating muscle progenitor cells, to thereby provide proliferated muscle progenitor cells.

In another preferred embodiment of a method for differentiating of the invention, said method for differentiating and/or said method for proliferating of a muscle progenitor cell followed by differentiating proliferated muscle progenitor cells, is an (entirely) serum-free method.

In another preferred embodiment of a method for differentiating of the invention, said muscle progenitor cell is a bovine muscle progenitor cell, preferably a bovine (myo)satellite cell.

In another preferred embodiment of a method for differentiating of the invention, said culturing of said muscle progenitor cell in said serum-free medium for differentiating is performed under conditions that allow for differentiation of said muscle progenitor cell into a myocyte, myotube and/or myofiber.

In another preferred embodiment of a method for differentiating of the invention, said method further comprises the step of:—incorporating said myocyte, myotube and/or myofiber into a meat product for human consumption, optionally in combination with adipocytes.

In another preferred embodiment of a method for differentiating of the invention, the serum-free medium for differentiating further comprises:
an epidermal growth factor (EGF) or a replacement thereof.

In another preferred embodiment of a method for differentiating of the invention, the serum-free medium for differentiating further comprises:
an albumin or a replacement thereof.

In another preferred embodiment of a method for differentiating of the invention, the serum-free medium for differentiating further comprises:
a source of glucose and/or a source of glutamine.

In another preferred embodiment of a method for differentiating of the invention, the serum-free medium for differentiating further comprises:
a source of iron and/or an iron transporter.

In another preferred embodiment of a method for differentiating of the invention, the serum-free medium for differentiating further comprises:
ascorbic acid or a derivative thereof.

In another preferred embodiment of a method for differentiating of the invention, the serum-free medium for differentiating further comprises:
sodium selenite.

In another preferred embodiment of a method for differentiating of the invention, the serum-free medium for differentiating further comprises:
ethanolamine.

In another preferred embodiment of a method for differentiating of the invention, the serum-free medium for differentiating further comprises:
insulin.

In another preferred embodiment of a method for differentiating of the invention, the serum-free medium for differentiating further comprises:
sodium bicarbonate.

In another preferred embodiment of a method for differentiating of the invention, the serum-free medium for differentiating comprises:—at least one differentiation inducer selected from the group consisting of a lysophosphatidic acid receptor 1 (LPAR1) agonist, a lysophosphatidic acid receptor 3 (LPAR3) agonist, an oxytocin receptor (OXTR) agonist, a glucagon receptor (GCGR) agonist and a lactate;—an epidermal growth factor (EGF) or a replacement thereof;—an albumin or a replacement thereof;—a source of glucose and a source of glutamine;—a source of iron or an iron transporter;—ascorbic acid or a derivative thereof;—sodium selenite; ethanolamine;—insulin; and—sodium bicarbonate; and optionally a Notch signaling pathway inhibitor.

In another preferred embodiment of a method for differentiating of the invention, all components in said serum-free medium for differentiating and/or serum-free medium for proliferating are animal-free.

In another aspect, the invention provides a serum-free medium for differentiating a muscle progenitor cell, wherein said medium is as defined in any one of the aspects and/or embodiments of a method for differentiating of the invention.

In a preferred embodiment of a method for differentiating of the invention or of a serum-free medium of the invention, wherein said lysophosphatidic acid receptor 1 (LPAR1) agonist such as lysophosphatidic acid is present, said lysophosphatidic acid receptor 1 (LPAR1) agonist is present in a concentration of 0.01-500 μM, preferably 0.5-50 μM, more preferably about 5 μM.

In another preferred embodiment of a method for differentiating of the invention or of a serum-free medium of the invention, wherein said lysophosphatidic acid receptor 3 (LPAR3) agonist such as lysophosphatidic acid is present, said lysophosphatidic acid receptor 3 (LPAR3) agonist is present in a concentration of 0.01-500 μM, preferably 0.5-50 μM, more preferably about 5 μM.

In another preferred embodiment of a method for differentiating of the invention or of a serum-free medium of the invention, wherein said oxytocin receptor (OXTR) agonist such as oxytocin is present, said oxytocin receptor (OXTR) agonist is present in a concentration of 0.01-1000 nM, preferably 5-500 nM, more preferably 50 nM.

In another preferred embodiment of a method for differentiating of the invention or of a serum-free medium of the invention, wherein said glucagon receptor (GCGR) agonist such as glucagon is present, said glucagon receptor (GCGR) agonist is present in a concentration of 0.01-100 μM, preferably 0.1-10 μM, preferably about 1 μM.

In another preferred embodiment of a method for differentiating of the invention or of a serum-free medium of the invention, wherein said lactate is present, said lactate is present in a concentration of 0.1-1000 mM, preferably 2-200 mM, more preferably about 10-20 mM.

In another preferred embodiment of a method for differentiating of the invention or of a serum-free medium of the invention, wherein said Notch signaling pathway inhibitor is present, said Notch signaling pathway inhibitor (such as DAPT) is present in a concentration of 0.01-1000 μM, for example 0.1-100 μM or 1-50 μM.

In another preferred embodiment of a method for differentiating of the invention or of a serum-free medium of the invention, a basal medium is present, preferably wherein said basal medium comprises DMEM and/or Ham's F12, more preferably either DMEM alone or DMEM and Ham's F12 medium in a ratio of 1:10-10:1, even more preferably either DMEM alone or DMEM and Ham's F12 medium in a 1:1 ratio. Alternatively, said basal medium may also comprise Alpha-MEM or M199. DMEM, Ham's F12 medium, alpha-MEM and M199 are examples of basal media. Basal media comprising modifications of these by replacement, reduction or elimination of any component may also be used. As an example, a basal medium such as DMEM may be supplemented with a pyruvate, a source of glucose and/or a source of glutamine.

In another preferred embodiment of a method for differentiating of the invention or of a serum-free medium of the invention, wherein a source of glucose and/or a source of glutamine is present, said source of glucose is present in a concentration of 0.01-10 g/l, preferably 0.1-4.5 g/l, and said source of glutamine is present in a concentration of 0.01-80 mM, preferably 0.1-8 mM.

In another preferred embodiment of a method for differentiating of the invention or of a serum-free medium of the invention, wherein albumin or a replacement thereof is present, said albumin or said replacement is present in a concentration of 0.01-50 g/l, preferably 0.05-5 g/l, more preferably 0.1-1 g/l.

In another preferred embodiment of a method for differentiating of the invention or of a serum-free medium of the invention, wherein a source of iron or an iron transporter such as transferrin is present, said source of iron or said iron transporter is present in a concentration of 0.1-1000 mg/l, preferably 1-100 mg/l, more preferably about 11 mg/l.

In another preferred embodiment of a method for differentiating of the invention or of a serum-free medium of the invention, wherein insulin is present, said insulin is present in a concentration of 0.1-400 mg/l, preferably 2-200 mg/l, more preferably about 19 mg/l.

In another preferred embodiment of a method for differentiating of the invention or of a serum-free medium of the invention, wherein sodium selenite is present, said sodium selenite is present in a concentration of 0.1-1000 μg/l, preferably 1-100 μg/l, more preferably about 14 μg/l.

In another preferred embodiment of a method for differentiating of the invention or of a serum-free medium of the invention, wherein ethanolamine is present in said medium, said ethanolamine is present in a concentration of 0.01-100 mg/l, more preferably 0.1-10 mg/l, even more preferably 2-5 mg/l.

In another preferred embodiment of a method for differentiating of the invention or of a serum-free medium of the invention, wherein said ascorbic acid or said derivative thereof is present, said ascorbic acid or said derivative is present in a concentration of 1-10000 mg/l, preferably 10-1000 mg/l or 50-500 mg/l, more preferably about 115 mg/l.

In another preferred embodiment of a method for differentiating of the invention or of a serum-free medium of the invention, wherein said epidermal growth factor (EGF) or a replacement thereof is present, said EGF or a replacement thereof is present in a concentration of 0.1-1000 μg/l, preferably 1-100 μg/l, more preferably about 10 μg/l.

In another preferred embodiment of a method for differentiating of the invention or of a serum-free medium of the invention, wherein said sodium bicarbonate is present, said sodium bicarbonate is present in a concentration of 1-10000 mg/l, preferably 50-5000 mg/l or 250-750 mg/l, more preferably about 543 mg/l.

In another aspect, the invention provides a composition comprising a serum-free medium for differentiating of the invention and a muscle progenitor cell and/or a partially or terminally differentiated cell such as a myoblast, myocyte, myotube and/or myofiber.

In another aspect, the invention provides a culture of myocytes, myotubes and/or myofibers obtainable by a method for differentiating of the invention.

In another aspect, the invention provides a meat product comprising myocytes, myotubes and/or myofibers obtainable by a method for differentiating of the invention, wherein said meat product optionally further comprises adipocytes, preferably bovine adipocytes.

In a preferred embodiment of a meat product of the invention, said meat product:—does not comprise inflammatory cells such as immune cells;—does not comprise antibiotics and/or antibiotic residues;—does not comprise red blood cells;—comprises lower levels of microbial contamination as compared to meat products obtained by animal slaughter;—does not comprise cartilage tissue; and/or—comprises lower levels of fibrous tissue as compared to meat products obtained by animal slaughter.

In another preferred embodiment of a meat product of the invention, said meat product:—does not comprise antibiotics and/or antibiotic residues; does not comprise red blood cells;—comprises lower levels of microbial contamination as compared to meat products obtained by animal slaughter; and/or—does not comprise cartilage tissue.

In another aspect, the invention provides a use of a serum-free medium of the invention for differentiating a progenitor cell. In the same manner, the invention provides a use of a differentiation inducer selected from the group consisting of a lysophosphatidic acid receptor 1 (LPAR1) agonist, a lysophosphatidic acid receptor 3 (LPAR3) agonist, an oxytocin receptor (OXTR) agonist, a glucagon receptor (GCGR) agonist, a lactate and a Notch signaling pathway inhibitor for differentiation of a progenitor cell, preferably myogenic differentiation of said progenitor cell, more preferably myogenic differentiation of said progenitor cell into a myoblast, myocyte, myotube and/or myofiber.

In another aspect, the invention provides a serum-free culture medium, comprising:—an epidermal growth factor (EGF) or a replacement thereof;—an albumin or a replacement thereof;—a source of glucose and/or a source of glutamine;—a source of iron or an iron transporter;—ascorbic acid or a derivative thereof;—sodium selenite;—ethanolamine;—insulin; and—sodium bicarbonate. The inventors established that with such a medium, even without the presence of one or more of the differentiation inducers described herein, it was possible to differentiate a progenitor cell to some extent (for instance wherein said progenitor cell was previously proliferated (expanded) under serum-free cell culture conditions), although the performance was considerably lower compared to the situation wherein a differentiation inducer as disclosed herein is included in the medium.

As one of skill in the art would readily understand, the source of glucose and/or the source of glutamine can be provided in the form of a basal medium such as a M199, alpha-MEM, DMEM and/or Ham's F12 medium, for instance DMEM alone or a DMEM and Ham's F12 medium in a ratio of 1:10 to 10:1, more preferably either DMEM alone or DMEM and Ham's F12 in a 1:1 ratio.

### DETAILED DESCRIPTION OF THE INVENTION

### Definitions

The term “serum-free”, as used herein, includes reference to a culture medium that is formulated in the absence of serum such as human serum or bovine serum. A serum-free medium may contain serum proteins such as serum albumin by way of supplementation of said serum albumin to said medium. However, preferably, all components of said medium are animal-free, i.e. that the components are not obtained from an animal but are for instance recombinantly produced. For instance, albumin is preferably recombinantly produced. The concentrations of the components of the serum-free medium of the invention can be adjusted and optimized for the culturing and differentiation, preferably myogenic differentiation, of progenitor cells such as bovine progenitor cells. As an example, a serum-free medium can comprise a serum-free basal medium that contains a source of glucose and/or a source of glutamine, or can comprise a serum-free basal medium that can be supplemented with a source of glucose and/or a source of glutamine. Preferably, in a serum-free medium of the invention (i.e. as described/disclosed herein), said differentiation inducer, said epidermal growth factor (EGF), said albumin, said basal medium, said source of glucose, said source of glutamine, said source of iron or said iron transporter, said ascorbic acid or said derivative thereof, said sodium selenite, said ethanolamine, said insulin and/or said sodium bicarbonate are present in an effective amount that allows for differentiation of a progenitor cell in culture, preferably wherein said progenitor cell in a previous culturing step has been proliferated or expanded (or originates or is derived from a culture of progenitor cells that was previously proliferated or expanded) under serum-free conditions such as with a serum-free medium for proliferation of progenitor cells. A non-limiting example of a serum-free medium for proliferation or expansion of muscle progenitor cells is provided in Example 3.

The term “culturing”, as used herein, includes reference to the differentiation or proliferation of progenitor cells such as bovine, ovine or porcine progenitor cells. In the context of the invention, this term preferably includes reference to the differentiation or proliferation of bovine progenitor cells. It should however be understood that a serum-free medium of the invention may also be employed to differentiate or proliferate other non-human, mammalian progenitor cells such as ovine and porcine (muscle) progenitor cells. Therefore, any embodiment described herein in relation to bovine progenitor cells, is also applicable to ovine (such as sheep) and porcine (such as pig) progenitor cells, i.e. progenitor cells of ovine or porcine origin. The term “culturing”, as used herein in relation to a method for differentiating a progenitor cell, includes reference to cell culture conditions that allow for differentiation, preferably myogenic differentiation, of a progenitor cell such as a (myo)satellite cell into a partially or terminally differentiated cell, preferably a myoblast, a myocyte, a myotube or a myofiber. The skilled person is well aware of suitable cell culture conditions that allow for differentiation of progenitor cells.

The term “differentiation”, as used herein, includes reference to induction of a differentiated phenotype in an undifferentiated cell such as a progenitor cell by co-culturing the undifferentiated cell in the presence of an inducer of cell differentiation. Differentiation is a developmental process whereby cells assume a specialized phenotype, e.g., acquire one or more characteristics or functions distinct from other cell types. In some cases, the differentiated phenotype refers to a cell phenotype that is at the mature endpoint in some developmental pathway (i.e. a terminally differentiated cell) such as a myocyte, a myotube or a myofiber. Preferably, the differentiation as referred to herein is a myogenic differentiation, i.e. a differentiation of a muscle progenitor cell (such as a muscle stem cell, for instance a satellite cell) into a myocyte or into a myotube or a myofiber.

The term “differentiation inducer”, as used herein, includes reference to an agent that induces, stimulates or activates differentiation, preferably myogenic differentiation. An example of myogenic differentiation is early phase myogenic differentiation, for instance early phase myogenic differentiation that induces or drives myogenic differentiation in the first 72 hours, including the first 24 hours, of differentiation.

The term “agonist”, as used herein, includes reference to a substance that binds to a receptor and activates the signaling pathway modulated by said receptor to thereby produce a response in a cell such as a progenitor cell. An agonist mimics the action of an endogenous ligand (and an agonist as used herein can be said endogenous ligand) that has an activating, stimulating or inductive effect on said receptor and/or said signaling pathway modulated by said receptor. Preferably, the agonist of a receptor is a lysophosphatidic acid receptor (LPAR) agonist, more preferably a lysophosphatidic acid receptor 1 (LPAR1) agonist or a lysophosphatidic acid receptor 3 (LPAR3) agonist; an oxytocin receptor (OXTR) agonist; or a glucagon receptor (GCGR) agonist.

Examples of known lysophosphatidic acid receptor 1 (LPAR1) agonist are lysophosphatidic acid (e.g. an oleoyl-L-α-lysophosphatidic acid, for instance in (sodium) salt form), N-palmitoyl serine phosphoric acid, N-acyl ethanolamide phosphate, 1-oleoyl-2-O-methyl-rac-glycerophospho-thionate isomers 2, 13 and 15, sn-2-aminooxy analogue 12b, alpha-fluoromethylene phosphonate, dialkyl thiophosphatidic acid, thiophosphate lipid analogue and oleoyl-thiophosphate. In embodiments, the lysophosphatidic acid receptor 1 (LPAR1) agonist as disclosed herein is one or more of the aforementioned lysophosphatidic acid receptor 1 (LPAR1) agonists, preferably said lysophosphatidic acid receptor 1 (LPAR1) agonist is a lysophosphatidic acid. Routine assays are available that allow a skilled person to assess whether an agent is a lysophosphatidic acid receptor 1 (LPAR1) agonist. Surface plasmon resonance (SPR) is an example of a widely used technique to measure association and dissociation rates for the binding kinetics between two species of chemicals, e.g., cell receptors and ligands.

Examples of known lysophosphatidic acid receptor 3 (LPAR3) agonists are lysophosphatidic acid (e.g. an oleoyl-L-α-lysophosphatidic acid, for instance in (sodium) salt form), N-palmitoyl serine phosphoric acid, N-acyl ethanolamide phosphate, 1-oleoyl-2-O-methyl-rac-glycerophospho-thionate and its isomers 2, 13 and 15, alpha-fluoromethylene phosphonate, alpha-hydroxymethylene phosphonate, dialkyl thiophosphatidic acid, dodecyl phosphate, thiophosphate lipid analogue and oleoyl-thiophosphate. In embodiments, the lysophosphatidic acid receptor 3 (LPAR3) agonist as disclosed herein is one or more of the aforementioned lysophosphatidic acid receptor 3 (LPAR3) agonists, preferably said lysophosphatidic acid receptor 3 (LPAR3) agonist is a lysophosphatidic acid. Routine assays are available that allow a skilled person to assess whether an agent is a lysophosphatidic acid receptor 1 (LPAR1) agonist. Surface plasmon resonance (SPR) is an example of a widely used technique to measure association and dissociation rates for the binding kinetics between two species of chemicals, e.g., cell receptors and ligands.

Examples of known oxytocin receptor (OXTR) agonists are peptide agonists such as oxytocin, carbetocin, vasopressin, desmopressin, demoxytocin, lipo-oxytocin-1 and merotocin, and non-peptide agonists such as TC OT 39, WAY-267464 and WAY 267464 dihydrochloride. In embodiments, the oxytocin receptor (OXTR) agonist as disclosed herein is one or more of the aforementioned oxytocin receptor (OXTR) agonists, preferably said oxytocin receptor (OXTR) agonist is oxytocin. Routine assays are available that allow a skilled person to assess whether an agent is an oxytocin receptor (OXTR) agonist. Surface plasmon resonance (SPR) is an example of a widely used technique to measure association and dissociation rates for the binding kinetics between two species of chemicals, e.g., cell receptors and ligands.

Examples of known glucagon receptor (GCGR) agonists are glucagon and peptide derivatives thereof such as glucagon 1-21 and glucagon 1-6, and also oxyntomodulin and NNC1702. In embodiments, the glucagon receptor (GCGR) agonist as disclosed herein is one or more of the aforementioned glucagon receptor (GCGR) agonists, preferably said glucagon receptor (GCGR) agonist is glucagon. Routine assays are available that allow a skilled person to assess whether an agent is a glucagon receptor (GCGR) agonist. Surface plasmon resonance (SPR) is an example of a widely used technique to measure association and dissociation rates for the binding kinetics between two species of chemicals, e.g., cell receptors and ligands.

The term “lactate”, as used herein, includes references to lactate as a free acid (lactic acid), lactate in salt form such as sodium lactate, or lactate in ionic form.

The term “Notch signaling pathway”, as used herein, includes reference to an evolutionary conserved signaling pathway that plays an integral role in development and tissue homeostasis in mammals. The Notch receptors and ligands contain single-pass transmembrane domains. Notch signaling is amongst others important in mediating communication between adjacent cells expressing the receptors and ligands. Notch receptors are heterodimeric proteins composed of extracellular and intracellular domains that are initially synthesized as a single polypeptide. Receptor-ligand interaction (i.e. Notch receptor activation) triggers a series of proteolytic cleavages of the Notch receptor polypeptide in which γ-secretase activity is involved. More specifically, γ-secretase activity provides for cleavage of the Notch intracellular domain (NICD; active form of the protein) which subsequently translocates to the nucleus to form a transcription factor complex.

The terms “γ-secretase” and “gamma-secretase” are used interchangeably herein.

The term “receptor”, as used herein, includes reference to a protein on the cell membrane or within the cytoplasm or cell nucleus that binds to a specific molecule (a ligand), such as a neurotransmitter, hormone, or other substance, and initiates the cellular response to the ligand. Ligand-induced changes in the behavior of receptor proteins result in cellular changes that constitute the biological actions of the ligands.

The term “Notch” or “Notch receptor”, as used herein, includes reference to one of the four mammalian, preferably bovine, Notch receptors, Notch1-4, and in particular, one of the bovine Notch 2 (e.g. UniProtKB-AOA3Q1MTU2) or Notch 3 (e.g. UniProtKB-E1BPT8) receptors.

The term “Notch signaling pathway inhibitor”, as used herein, includes reference to a substance such as a compound, protein, peptide or other molecule that inhibits or antagonizes Notch signaling, e.g. by inhibiting or antagonizing Notch receptor activation. Preferably, said substance inhibits or antagonizes Notch signaling pathway activation, stimulation or induction, e.g. by inhibiting gamma-secretase activity using suitable inhibitors such as DAPT. A preferred Notch signaling pathway inhibitor is a gamma-secretase inhibitor. Assays to screen compounds for Notch signaling pathway inhibitory activity are routinely available to the skilled person. As an example, the Notch Reporter Assay Kit (Genway Biotech, GWB-PS79B7) can be used to monitor the activity of Notch signaling pathway in cultured cells and can thus be used to screen for Notch signaling pathway inhibitors.

The term “progenitor cell”, as used herein, includes reference to a cell that is committed to differentiate into a specific type of cell or to form a specific type of tissue. The term “progenitor cell” may include reference to a cell that is able to differentiate into a more specialized cell, such as multipotent stromal cells (mesenchymal stem cells) with the capacity for self-renewal and multipotential differentiation into inter alia myocytes (muscle cells). Preferably, the progenitor cell is a muscle progenitor cell. A progenitor cell can be a tissue-derived progenitor cell, derived from a wildtype (e.g. domestic cow, sheep or pig) or a transgenic animal (e.g. transgenic cow, sheep or pig). The progenitor cell itself can be genetically modified or can be not genetically modified. For example, the progenitor cell can be an induced pluripotent stem cell (iPS) generated from a cell of bovine, ovine or porcine origin. Preferably, the progenitor cell is a muscle tissue- or adipose tissue-derived progenitor cell that is not genetically modified. A progenitor cell as referred to herein can be a progenitor cell that is expanded (in population size) in a proliferation medium, for instance a serum-free proliferation medium, by cell culture without differentiating said progenitor cell.

The term “bovine progenitor cells”, as used herein, includes reference to a bovine cell that is committed to differentiate into a specific type of bovine cell or to form a specific type of bovine tissue. The term “bovine progenitor cell” may include reference to multipotent bovine stromal cells (mesenchymal stem cells) with the capacity for self-renewal and multipotential differentiation into inter alia myocytes (muscle cells). Preferably, the bovine progenitor cell is a bovine muscle progenitor cell. A bovine progenitor cell can be a tissue-derived bovine progenitor cell, derived from a wildtype (e.g. domestic cow) or transgenic animal (e.g. transgenic cow). The bovine progenitor cell itself can be genetically modified or can be not genetically modified. For example, the bovine progenitor cell can be an induced pluripotent stem cell (iPS) generated from a bovine cell. Preferably, the bovine progenitor cell is a tissue-derived bovine progenitor cell that is not genetically modified.

The term “bovine”, as used herein, includes reference to animals belonging to the family of Bovidae, including the genus Bos. The term “bovine”, as used in aspects and embodiments described herein, may be replaced by the term “bovid”. The term “bovid” can be used to refer to any animal in the family of Bovidae. The family of Bovidae includes bison, buffalo, antelopes, wildebeest, impala, gazelles, sheep, goats, muskoxen, and cattle (such as cows), including domestic cattle. Especially preferred bovine species are Bos taurus (cow).

The term “ovine”, as used herein, includes reference to any animal that belongs to the genus of Ovis, which includes the species Ovis aries. The term includes reference to sheep, which can be a domesticated or a wild species.

The term “porcine”, as used herein, includes reference to any animal in the family of Suidae, which includes the subfamily Suinae and the genus Sus. The term includes reference to pigs, which can be a domesticated or a wild species.

The term “muscle progenitor cell”, as used herein, can be used interchangeably with the term “muscle stem cell” or “myogenic progenitor (cell)”. These terms include reference to adult stem cells, present in tissue such as skeletal muscle tissue, which are multipotent and which can self-renew and are capable of giving rise to muscle cells such as skeletal muscle cells. The term also includes reference to fat (tissue)-derived muscle progenitor cells, which are progenitor cells that are present in fat tissue and which can give rise to muscle cells such as skeletal muscle cells. The term “muscle progenitor cell” can also be referred to as “muscle cell progenitor”. A preferred muscle progenitor cell is a bovine muscle progenitor cell such as a bovine myosatellite cell. Preferably, the muscle progenitor cell, more preferably the myosatellite cell, is a (skeletal) muscle tissue-derived progenitor cell. Such a cell can be obtained by direct isolation from an animal or can be obtained after proliferation (expansion) in a proliferation medium, preferably a serum-free proliferation medium. Such a cell can be genetically modified or not genetically modified, preferably not genetically modified. Progenitor cells from the muscle can be isolated based on their positive expression of CD29 as previously described (Ding et al., Sci. Rep. 17(8): 10808 (2018)). Such progenitor cells can be expanded in population size by using a proliferation medium that does not induce differentiation of said progenitor cells.

The term “myosatellite cell”, as used herein, includes reference to a small multipotent cell and can be found in mature muscle tissue. Myosatellite cells are precursors to skeletal muscle cells, able to give rise to satellite cells or differentiated skeletal muscle cells. They are precursor cells that can be obtained from muscle tissue. They have the potential to provide additional myonuclei to their parent muscle fiber, or return to a quiescent state. More specifically, upon activation, satellite cells can re-enter the cell cycle to proliferate or differentiate into myoblasts and ultimately into myocytes which will fuse forming myotubes. Myosatellite cells are generally located between the basement membrane and the sarcolemma of a muscle fibers. Myosatellite cells generally express a number of distinctive genetic markers. Most satellite cells express PAX7 and PAX3.

The term “iron”, as used herein, includes reference to a salt containing iron such as ferric nitrate and ferrous sulfate.

The term “source of”, as used herein, includes reference to a medium component that is provided as a precursor of said medium component, or is provided as the medium component as such. The skilled person is well aware of suitable precursors for medium components as described herein. For instance, L-alanyl-L-glutamine or glutamine can be used as a source of glutamine, α-linolenic acid can be used as a source of fatty acids, and glucose can be used as a source of glucose.

The term “growth factor” as used herein, includes reference to a biomolecule, namely a protein regulating aspects of cellular function, such as differentiation. Within the group of said proteins, epidermal growth factor (EGF) is included. Preferably, the growth factors and/or hormones mentioned herein are of human origin, and are preferably recombinantly produced. In general, where reference is made to a growth factor or hormone or other protein, such a protein can be a wildtype protein or a protein that is mutated or otherwise modified as compared to its wildtype equivalent such as its human wildtype equivalent.

The term “replacement”, as used herein, includes reference to any compound or group of compounds or combination of compounds such as a peptide, protein and/or small molecule that can serve as a replacement for a certain medium component without negatively affecting muscle progenitor cell differentiation. In other words, compared to the situation wherein a certain medium component is present in a serum-free medium of the invention, replacement of said medium component does not negatively affecting muscle progenitor cell differentiation.

### Serum-Free Medium of the Invention

The invention provides a serum-free medium for differentiating a muscle progenitor cell, wherein said serum-free medium comprises:—at least one differentiation inducer selected from the group consisting of a lysophosphatidic acid receptor 1 (LPAR1) agonist, a lysophosphatidic acid receptor 3 (LPAR3) agonist, an oxytocin receptor (OXTR) agonist, a glucagon receptor (GCGR) agonist, a lactate and a Notch signaling pathway inhibitor. LPAR1 is preferably a bovine LPAR1 such as identified by UniProtKB-Q28031. LPAR3 is preferably a bovine LPAR3 such as identified by UniProtKB-F1MX11. OXTR is preferably a bovine OXTR such as identified by UniProtKB-P56449. GCGR is preferably a bovine GCGR such as identified by UniProtKB-E1BKB6.

In embodiments, at least one of the differentiation inducers as disclosed herein can be employed in a serum-free medium of the invention in combination with one or more further differentiation inducer as disclosed herein. For instance, combinations of (i) an LPAR1 agonist or an LPAR3 agonist, and an OXTR agonist, (ii) an LPAR1 agonist or an LPAR3 agonist, and a GCGR agonist, (iii) an LPAR1 agonist or an LPAR3 agonist, and a lactate, (iv) an OXTR agonist and a GCGR agonist, (v) an OXTR agonist and a lactate, (vi) a GCGR agonist and a lactate, and (vii) an LPAR1 agonist and an LPAR3 agonist, each of (i)-(vii) optionally in combination with a Notch signaling pathway inhibitor, are envisaged. In other embodiments, at least three, at least four or at least five of the differentiation inducers as disclosed herein can be employed in a serum-free medium of the invention. For instance, an example of at least three differentiation inducers is a combination of at least LPAR1 (or an LPAR3) agonist, an OXTR agonist and a GCGR agonist, optionally in combination with a Notch signaling pathway inhibitor. For such a combination, myotube stabilization was observed (data not shown).

Preferably, said serum-free medium comprises (i) at least one differentiation inducer selected from the group consisting of a lysophosphatidic acid receptor 1 (LPAR1) agonist, a lysophosphatidic acid receptor 3 (LPAR3) agonist, an oxytocin receptor (OXTR) agonist, a glucagon receptor (GCGR) agonist and a lactate, and (ii) a Notch signaling pathway inhibitor. Such a combination allows for further improved myogenic differentiation of muscle progenitor cells.

Preferably, the differentiation inducer that is a lysophosphatidic acid receptor 1 (LPAR1) agonist is selected from the group formed by lysophosphatidic acid, N-palmitoyl serine phosphoric acid, N-acyl ethanolamide phosphate, 1-oleoyl-2-O-methyl-rac-glycerophospho-thionate isomers 2, 13 and 15, sn-2-aminooxy analogue 12b, alpha-fluoromethylene phosphonate, dialkyl thiophosphatidic acid, thiophosphate lipid analogue, and oleoyl-thiophosphate. Most preferably, the LPAR1 agonist is a lysophosphatidic acid. This inducer can be obtained from Sigma Aldrich Cat. Nr. L7260. Preferably, the LPAR1 agonist is present in a serum-free medium of the invention in a concentration of 0.01-500 μM, preferably 0.5-50 μM, more preferably about 5 μM. Lysophosphatidic acid can already be comprised in a basal medium, or can be supplemented to a basal medium.

The term “lysophosphatidic acid”, as used herein in relation to a differentiation inducer, includes reference all its forms such as its free acid (protonated) form, conjugate base (non-protonated) form, and salt form (such as lysophosphatidic acid (sodium) salt.

Preferably, the differentiation inducer that is a lysophosphatidic acid receptor 3 (LPAR3) agonist is selected from the group formed by lysophosphatidic acid, N-palmitoyl serine phosphoric acid, N-acyl ethanolamide phosphate, 1-oleoyl-2-O-methyl-rac-glycerophospho-thionate and its isomers 2, 13 and 15, alpha-fluoromethylene phosphonate, alpha-hydroxymethylene phosphonate, dialkyl thiophosphatidic acid, dodecyl phosphate, thiophosphate lipid analogue and oleoyl-thiophosphate. Most preferably, the LPAR3 agonist is a lysophosphatidic acid. This inducer can be obtained from Sigma Aldrich Cat. Nr. L7260. Preferably, the LPAR3 agonist is present in a serum-free medium of the invention in a concentration of 0.01-500 μM, preferably 0.5-50 μM, more preferably about 5 μM. Lysophosphatidic acid can already be comprised in a basal medium, or can be supplemented to a basal medium.

Preferably, the differentiation inducer that is an oxytocin receptor (OXTR) agonist is selected from the group formed by oxytocin, carbetocin, vasopressin, desmopressin, demoxytocin, lipo-oxytocin-1, merotocin, TC OT 39, WAY-267464 and WAY 267464 dihydrochloride. Most preferably, the OXTR agonist is oxytocin. This inducer can be obtained from Sigma Aldrich Cat. Nr. 06379. Preferably, the OXTR agonist is present in serum-free medium of the invention in a concentration of 0.01-1000 nM, preferably 5-500 nM, more preferably 50 nM. Oxytocin can already be comprised in a basal medium, or can be supplemented to a basal medium.

Preferably, the differentiation inducers that is a glucagon receptor (GCGR) agonist is selected from the group formed by glucagon and any one of the peptide derivatives thereof such as glucagon 1-21 and glucagon 1-6, and also oxyntomodulin and NNC1702. Most preferably, the GCGR agonist is glucagon. This inducer can be obtained from Sigma Aldrich Cat. Nr. G2044. Preferably, the glucagon agonist is present in serum-free medium of the invention in a concentration of 0.01-100 μM, preferably 0.1-10 μM, preferably about 1 μM. Glucagon can already be comprised in a basal medium, or can be supplemented to a basal medium.

Preferably, the lactate is obtained from SigmaAldrich Cat. Nr. 71718. The lactate can be supplemented to a serum-free medium in the form of a salt such as a sodium salt. It is preferred that the lactate is present in a serum-free culture medium of the invention in a concentration of 0.1-1000 mM, preferably 2-200 mM, more preferably about 10-20 mM. Lactate can already be comprised in a basal medium, or can be supplemented to a basal medium.

Preferably, the differentiation inducer that is a Notch signaling pathway inhibitor is a gamma-secretase inhibitor. In embodiments, the gamma-secretase inhibitor is selected from the group formed by DAPT (CAS 208255-80-5), E 2012 (CAS 870843-42-8), L685458 (CAS 292632-98-5), R04929097 (CAS 847925-91-1) and LY-411575 (CAS 209984-57-6). Preferably, the gamma-secretase inhibitor is DAPT. DAPT can be obtained from Abcam (#ab120633). The Notch signaling pathway inhibitor can be present in a serum-free medium for differentiating a muscle progenitor cell as disclosed herein in a concentration of 0.01-1000 μM, for example 0.1-100 μM or 1-50 μM.

It is preferred that a serum-free medium of the invention further comprises an epidermal growth factor (EGF) or a replacement thereof.

Preferably, said epidermal growth factor (EGF) is a recombinant (i.e. recombinantly produced) human or bovine epidermal growth factor (EGF), preferably bovine epidermal growth factor (EGF). EGF can be obtained from Peprotech Cat. Nr. AF-100-15. It is preferred that the EGF is present in a serum-free medium of the invention in a concentration of 0.1-1000 μg/l, preferably 1-100 μg/l, more preferably about 10 μg/l. EGF can already be comprised in a basal medium, or can be supplemented to a basal medium.

Preferably, the serum-free medium of the invention further comprises an albumin or a replacement thereof. Preferably, the albumin is a human albumin, such as a recombinant human albumin, for instance obtained from Biorbyt (orb419911). It is preferred that the albumin is present in a serum-free culture medium of the invention in a concentration of 0.01-50 g/I, preferably 0.05-5 g/I, more preferably 0.1-1 g/I. Albumin can already be comprised in a basal medium, or can be supplemented to a basal medium.

It is preferred that a serum-free medium of the invention further comprises a source of glucose and/or a source of glutamine. A preferred source of glucose comprises glucose, a preferred source of glutamine comprises glutamine or L-alanyl-L-glutamine. Glutamine or L-alanyl-L-glutamine can be obtained from Thermo Fisher Scientific (Gibco® GlutaMAX™ Cat nr: 35050061).

The source of glucose can be present in a serum-free medium of the invention in a concentration of 0.01-10 g/l, preferably 0.1-4.5 g/l, and said source of glutamine can be present in a serum-free medium of the invention in a concentration of 0.01-80 mM, preferably 0.1-8 mM. The source of glucose and/or a source of glutamine can already be comprised in a basal medium, or can be supplemented to a basal medium.

It is preferred that a serum-free medium of the invention further comprises a source of iron or an iron transporter, preferably transferrin. The source of iron or the iron transporter can be present in a concentration of 0.1-1000 mg/l, preferably 1-100 mg/l, more preferably about 11 mg/l. Iron or an iron transporter can already be comprised in a basal medium, or can be supplemented to a basal medium. An iron transporter such as transferrin can be obtained from Peprotech Biogems Cat. Nr. 10-366.

Preferably, a serum-free medium of the invention further comprises ascorbic acid or a derivative thereof such as L-ascorbic acid 2-phosphate. L-ascorbic acid 2-phosphate can be obtained from Sigma Aldrich Cat. Nr. A8960. Ascorbic acid or a derivative thereof such as L-ascorbic acid 2-phosphate can be present in a serum-free medium of the invention in a concentration of 1-10000 mg/l, preferably 10-1000 mg/l or 50-500 mg/l, more preferably about 115 mg/l. Ascorbic acid or a derivative thereof can already be comprised in a basal medium, or can be supplemented to a basal medium.

It is preferred that a serum-free medium of the invention further comprises (sodium) selenite. (Sodium) selenite can be obtained from Sigma Aldrich Cat. Nr. S5261. Sodium selenite can be present in a serum-free medium of the invention in a concentration of 0.1-1000 μg/l, preferably 1-100 μg/l, more preferably about 14 μg/l. Sodium selenite, or its cation and anion in a liquid solution, can already be comprised in a basal medium, or can be supplemented to a basal medium for instance in the form of its salt sodium selenite.

It is preferred that a serum-free medium of the invention further comprises ethanolamine. Ethanolamine can be obtained from Sigma Aldrich Cat. Nr. E9508. Ethanolamine can be present in a serum-free medium of the invention in a concentration of 0.01-100 mg/l, preferably 0.1-10 mg/l, more preferably about 4 mg/l. Ethanolamine can already be comprised in a basal medium, or can be supplemented to a basal medium.

It is preferred that a serum-free medium of the invention further comprises an insulin. Said insulin is preferably a human insulin, more preferably a recombinantly produced human insulin. Recombinantly produced human insulin can be obtained from Sigma Aldrich (91077C). Insulin can be present in a serum-free medium of the invention in a concentration of 0.1-400 mg/l, preferably 2-200 mg/l, more preferably about 19 mg/l. Insulin can already be comprised in a basal medium, or can be supplemented to a basal medium.

In a preferred embodiment, said insulin, transferrin, ethanolamine and selenite can be provided, such as supplemented to a basal medium, in the form of a Insulin-Transferrin-Selenium-Ethanolamine (ITSE) supplement. Said basal medium may in addition further comprise a pyruvate, a source of glutamine and/or a source of glucose. In addition, or alternatively, a basal medium may further comprise an (essential) amino acid supplement (such as MEM AA solution 50×(Gibco catalog number 11130051), a soy hydrolysate and optionally a PSA (Penicillin Streptomycin Amphotericin) supplement.

It is preferred that a serum-free medium of the invention further comprises sodium bicarbonate. Sodium bicarbonate can be obtained from Sigma Aldrich Cat. Nr. S5761. Sodium bicarbonate can be present in a serum-free medium of the invention in a concentration of 1-10000 mg/l, preferably 50-5000 mg/l or 250-750 mg/l, more preferably about 543 mg/l. Sodium bicarbonate, or its cation and anion in a liquid solution, can already be comprised in a basal medium, or can be supplemented to a basal medium for instance in the form of its salt sodium bicarbonate.

More preferably, a serum-free medium of the invention comprises:—at least one differentiation inducer selected from the group consisting of a lysophosphatidic acid receptor 1 (LPAR1) agonist, a lysophosphatidic acid receptor 3 (LPAR3) agonist, an oxytocin receptor (OXTR) agonist, a glucagon receptor (GCGR) agonist and a lactate;—an epidermal growth factor (EGF) or a replacement thereof;—an albumin or a replacement thereof;—a source of glucose and a source of glutamine;—a source of iron or an iron transporter;—ascorbic acid or a derivative thereof;—(sodium) selenite;—ethanolamine:—insulin; and (sodium) bicarbonate; and optionally a Notch signaling pathway inhibitor. In the same manner, a preferred serum-free medium of the invention comprises: L-Ascorbic acid 2-phosphate, (sodium) selenite, ethanolamine, insulin, transferrin, (sodium) bicarbonate, albumin, EGF; alpha-MEM or M199 or DMEM alone or DMEM/F12 basal medium; and one or more of lactate, lysophosphatidic acid, oxytocin or glucagon; and optionally a gamma-secretase inhibitor such as DAPT. Even more preferably, a serum-free medium of the invention comprises: L-Ascorbic acid 2-phosphate in a concentration of about 115 μg/ml, (sodium) selenite in a concentration of about 0.014 μg/ml, ethanolamine in a concentration of about 4 μg/ml, insulin in a concentration of about 19 μg/ml, transferrin in a concentration of about 11 μg/ml, (sodium) bicarbonate in a concentration of about 543 μg/ml, albumin in a concentration of about 0.5 mg/ml, EGF in a concentration of about 10 ng/ml, DMEM alone or DMEM/F12 basal medium, and one or more of lactate in a concentration of about 10-20 mM, lysophosphatidic acid in a concentration of about 0.5-10 μM, oxytocin in a concentration of 10-200 nM, or glucagon in a concentration of 0.1-2 μM; and optionally a gamma-secretase inhibitor such as DAPT.

Preferably, in a serum-free medium of the invention, all components are animal-free. In other words, the medium components are not obtained from an animal but are for instance recombinantly produced. The invention also provides a composition comprising a serum-free medium of the invention, and a cell such as a muscle progenitor cell and/or a partially or terminally differentiated cell obtained from said progenitor cell.

In embodiments, a composition of the invention is a cell culture. Most preferably, the progenitor cell is a bovine muscle progenitor cell such as a bovine myosatellite cell. Preferably, the progenitor cell is muscle-tissue derived and not genetically modified. Such a progenitor cell can be a cell that is produced through expansive/proliferative cell culture in a proliferation/expansion medium. Most preferably, such a proliferation/expansion medium was also a serum-free medium. An example of a partially differentiated cell obtained from a progenitor cell is a myoblast. An example of a terminally differentiated cell obtained from a progenitor cell is a myocyte, a myotube or a myofiber.

Preferably, in a serum-free medium of the invention or in a composition of the invention, the progenitor cell is a bovine, ovine or porcine progenitor cell, preferably a bovine, ovine or porcine muscle progenitor cell.

The invention also provides a method for producing a serum-free medium of the invention, comprising the step of—adding the constituents or components of a serum-free medium of the invention as disclosed herein to a basal medium, preferably a liquid basal medium such as a liquid basal medium comprising alpha-MEM or M199 or DMEM or Ham's F12 medium, preferably either DMEM alone or DMEM and Ham's F12 medium for instance in a ratio of 1:10-10:1, more preferably either DMEM alone or DMEM and Ham's F12 medium in a 1:1 ratio, respectively.

### Methods for Differentiating a Progenitor Cell.

The invention also provides a method for differentiating a muscle progenitor cell, comprising the step of:—culturing a muscle progenitor cell in a serum-free medium for differentiating a muscle progenitor cell, wherein said serum-free medium comprises—at least one differentiation inducer selected from the group consisting of a lysophosphatidic acid receptor 1 (LPAR1) agonist, a lysophosphatidic acid receptor 3 (LPAR3) agonist, an oxytocin receptor (OXTR) agonist, a glucagon receptor (GCGR) agonist, a lactate and a Notch signaling pathway inhibitor.

Preferably, the progenitor cell is cultured in a serum-free medium as disclosed herein above. The step of culturing is under culture conditions that allow for differentiation of said progenitor cell, preferably myogenic differentiation of said progenitor cell, more preferably myogenic differentiation of said progenitor cell into a myoblast, myocyte, myotube and/or myofiber. The skilled person is aware of appropriate culture conditions under which myogenic differentiation can be induced using a serum-free differentiation medium as disclosed herein. For instance, bovine muscle progenitor cells isolated from a bovine muscle tissue previously propagated in a serum-free proliferation medium for at least 8 population doublings can be plated in a serum-free proliferation medium onto a Matrigel Matrix (356230 from Corning)-coated cell culture vessel at a density of 37500 cells/cm². Differentiation can subsequently be induced by changing the medium to a serum-free differentiation medium of the invention about 24 h later.

Preferably, in a method for differentiating of the invention, the progenitor cell is a bovine, ovine or porcine progenitor cell, preferably a bovine, ovine or porcine muscle progenitor cell. Even more preferably, in a method for differentiating of the invention, said bovine, ovine or porcine muscle progenitor cell is differentiated into a bovine, ovine or porcine myoblast, myocyte, myotube and/or myofiber.

Preferably, in a method for differentiating of the invention, progenitor cells have not been cultured, in any stage, with a serum-containing medium, more preferably have not been cultured with a medium that contains animal components, i.e. components that are obtained or isolated from an animal. Therefore, in a preferred embodiment, a method for differentiating of the invention is part of an (entirely) serum-free cell culture method for the production of cultured meat for human consumption.

A method for differentiating of the invention may further comprise a step of: isolating or purifying a bovine, ovine or porcine myoblast, myocyte, myotube and/or myofiber from said culture medium.

More preferably, said method for differentiating of the invention may further comprise a step of—incorporating said myocyte, myotube and/or myofiber into a meat product for human consumption, optionally in combination with adipocytes. The meat product for human consumption is preferably a cell-culture-based meat product, such as a cell-culture-based minced meat product. Further, in said step of incorporating said myocyte, myotube and/or myofiber into a meat product for human consumption, said myocyte, myotube and/or myofiber can be combined with adipocytes into said meat product.

The invention also provides a culture of myocytes, myotubes and/or myofibers obtainable by a method for differentiating of the invention.

The invention also provides a meat product comprising myocytes, myotubes and/or myofibers obtainable by a method for differentiating of the invention, said meat product optionally further including adipocytes. The meat product for human consumption of the invention is preferably a cell-culture-based meat product, such as a cell-culture-based minced meat product, and preferably is devoid of serum components (preferably devoid of components originating from serum) such as fetal bovine serum components.

For the purpose of clarity and a concise description, features are described herein as part of the same or separate embodiments, however, it will be appreciated that the disclosure includes embodiments having combinations of all or some of the features described.

The content of the documents referred to herein is incorporated by reference.

### FIGURE LEGENDS

FIG. 1 shows myogenic differentiation of bovine muscle progenitor cells into myocytes in a serum-free myogenic differentiation medium as disclosed herein containing the agonists (differentiation inducers) of the identified overexpressed receptors or lactate. Before myogenic differentiation, the cells were expanded in a serum-free proliferation medium.

FIG. 2 shows myogenic differentiation of bovine muscle progenitor cells into myocytes in a serum-free myogenic differentiation medium as disclosed herein, whereby said agonists (differentiation inducers) of the identified overexpressed receptors or lactate where added in a range of concentrations. In FIG. 2, LPA refers to lysophosphatidic acid, OT refers to oxytocin and GCG refers to glucagon.

FIG. 3.

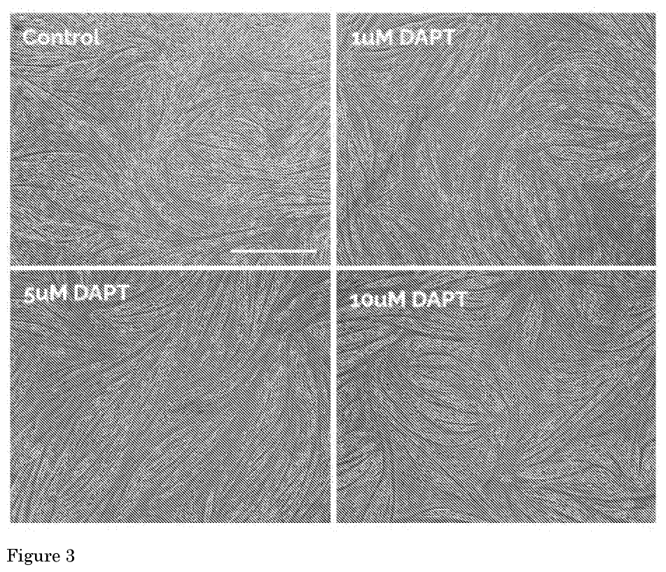

Notch signaling inhibits myogenic differentiation in a subpopulation of satellite cells, which can be improved by the addition of an inhibitor of Notch signaling such as a gamma secretase inhibitor like DAPT. More specifically, FIG. 3 shows myogenic differentiation of bovine muscle progenitor cells into myocytes in a serum-free myogenic differentiation medium with the addition of DAPT in a range of concentration (1 μM, 5 uM and 10 uM) in comparison to the control serum-free myogenic differentiation medium in which DAPT is absent.

### EXAMPLES

### Example 1. RNA Expression During Myogenic Differentiation

### Materials and Methods

### Myogenic Differentiation

Bovine satellite cells were differentiated on Matrigel coated flasks by seeding 5×10⁵ cells/cm² in growth medium containing 20% bovine serum. After 24 h, differentiation was induced by decreasing serum concentration from 20% to 2%.

### RNA Isolation

RNA lysates were harvested from tissue culture samples by directly adding TRK lysis buffer onto the samples after removing culture media and washing with PBS. The RNA was purified by using the Omega MicroElute Total RNA Kit (Bio-Rad) following the supplier's protocol for tissue culture. RNA concentrations were determined by nanospectrometry.

Pre-sequencing quality control was performed using a bioanalyzer. The library was prepared using the TruSeq stranded mRNA kit (Illumina) and sequenced with 12 samples per run on a high-output 75 bp NextSeq flowcell. On average, 37.0*10\hat{ }6 (±7.3*10\hat{ }6) of aligned reads per samples were obtained.

### Read Alignment and Quantification

The single-end reads were aligned to the reference genome tau9 Bos_taurus.ARS-UCD1.2.98.gtf using STAR aligner (Dobin et al., Bioinformatics 29, 15-21 (2013)) and assigned to genes using FeatureCounts function of the Rsubread package (Liao et al., Bioinforma. Oxf. Engl., 30:923-930 (2014)). On average, 78.86% (±1.04%) of reads were uniquely assigned to genes.

### Quality Control and Normalization

Next, a DGEList-object was created using the obtained count matrix and gene meta information from the Btaurus_gene_ensembl data set (Yates et al., Ensembl 2020. Nucleic Acids Res. 48, D682-D688 (2020); Robinson et al., Bioinforma. Oxf. Engl., 26, 139-140 (2010)). Low-abundance genes (min total count 15 below, expressed in at least 3 of 4 replicates) were removed and normalization factors were calculated using the trimmed-mean of M-values (TMM) method in the NormFactor function of edgeR (Robinson et al., Genome Biol., 11, R25 (2010); Anders et al., Genome Biol. 11, R106 (2010)). Finally, counts per million (cpm) and reads-per-kilobase per million were computed based on the normalized library sizes.

### Dimensionality Reduction and Differential Expression Analysis

Principal component analysis was performed using the 500 most variable genes based on the variance of RPKMs. Differential expression analysis was performed for each gene between each day of differentiation by empirical eBayes moderation towards a common value with a lfc threshold of log(1.2) (McCarthy et al., Nucleic Acids Res. 40, 4288-4297 (2012); Ritchie et al., Nucleic Acids Res. 43, e47 (2015)). Genes were considered differentially expressed (DE) above a log-FC cutoff of 1 and a FDR below 5% and visualized in respective volcano plots. A heatmap showing the z-values of the 1000 most differentially expressed genes between DO and D1 was constructed in which samples were clustered using the Ward's minimum variance method with euclidean distances (Ward, J. Am. Stat. Assoc. 58, 236-244 (1963)). Finally, over-represented gene ontology (GO) terms were computed for both upregulated and downregulated DE genes (Ashburner et al., The Gene Ontology Consortium. Nat. Genet. 25, 25-29 (2000); The Gene Ontology Consortium, Nucleic Acids Res. 47, D330-D338 (2019)).

### Results

The RNA sequencing experiments provided for the identification of receptors that are upregulated in the early phase of myogenic differentiation, which allows for the identification of myogenic differentiation inducers. Table 1 identifies, by analysis of RNA sequencing data obtained during early phase (day0/day1) myogenic differentiation under serum-containing conditions, upregulated genes coding for membrane receptors, with an indication of respective agonists to be used as inducers under serum-free conditions.

TABLE 1
Identification of differentiation inducers from
RNAseq during serum-containing differentiation.

| Ratio | | | | |
|---|---|---|---|---|
| Day 0/Day 1 | Upregulation | | | |
| (log₂)* | (Ratio) | Gene | Name | Agonist |
| −4.693 | 25.9 | LPAR1 | lyso- | lyso- |
| | | | phosphatidic | phosphatidic |
| | | | acid receptor 1 | acid |
| −3.559 | 11.8 | LPAR3 | lyso- | lyso- |
| | | | phosphatidic | phosphatidic |
| | | | acid receptor 3 | acid |
| −3.429 | 22.6 | OXTR | oxytocin | oxytocin |
| | | | receptor | |
| −2.051 | 4.1 | GCGR | glucagon | glucagon |
| | | | receptor | |

*a negative value means upregulation on day 1 compared with day 0.

### Example 2. Production of a Serum-Free Medium of the Invention

An exemplary serum-free medium for differentiation of a progenitor cell as disclosed herein was prepared as follows.

The DMEM/F12 1:1 or 10:1 or 1:0 or alpha-MEM or M199 medium was supplemented with albumin (recombinant human albumin from Richcore) at 0.5 mg/ml, insulin (recombinant human insulin, 10-365 from Peprotech) at 19.4 ug/ml, transferrin (recombinant human transferrin, 10-366 from Peprotech) at 10.7 ug/ml, sodium selenite (S5261 from SigmaAldrich) at 0.014 ug/ml, Ethanolamine from Sigma Aldrich cat nr E9508 at 4 ug/ml, ascorbic acid (L-ascorbic acid 2-phosphate sesquimagnesium salt hydrate, A8960 from SigmaAldrich) at 115.22 ug/ml, EGF (recombinant human EGF, AF-100-15 from Peprotech) at 10 ng/ml and one of the following inducers: lactate (Sodium L-lactate, 71718 from SigmaAldrich) at 10 mM, LPA (Oleoyl-L-α-lysophosphatidic acid sodium salt, L7260 from SigmaAldrich) at 5 uM, oxytocin (06379 from SigmaAldrich) at 50 nM, or glucagon (G2044 from SigmaAldrich) at 1 uM.

### Example 3. Myogenic Differentiation of Progenitor Cells

### Materials and Methods

Bovine muscle progenitor cells were isolated from a bovine muscle tissue (Bos taurus) and sorted based on their positive expression of CD29 as previously described (Ding et al., Sci. Rep., 17(8): 10808 (2018)). Muscle progenitor cells were propagated in a serum-free proliferation medium for at least 8 population doublings prior to differentiation. Briefly, the cells were seeded at a density of 5000 cells/cm² in a serum-free proliferation medium (albumin (5 mg/ml), somatotropin (2 ng/ml), L-Ascorbic acid 2-phosphate (50 μg/ml), hydrocortisone (36 ng/ml), α-linolenic acid (1 μg/ml), insulin (10 μg/ml), transferrin (5.5 μg/ml), sodium selenite (0.0067 μg/ml), ethanolamine (2 μg/ml), L-alanyl-L-glutamine or glutamine (2 mM), IL-6 (5 ng/ml), FGF2 also referred to as bFGF (10 ng/ml), IGF1 (100 ng/ml), VEGF (10 ng/ml), HGF (5 ng/ml), PDGF-BB (10 ng/ml) and DMEM/F12 basal medium) in an appropriate collagen-coated cell culture vessel. The cells were passaged upon reaching 90% confluency by rinsing once with phosphate buffer saline (PBS, 20012027 from ThermoFischer Scientific) followed by the addition of trypsin (25200072 from ThermoFischer Scientific). Once the cells were detached, trypsin was neutralised by the addition of trypsin inhibitor from Glycine max (T6522 from Sigma Aldrich), the cells collected into PBS and centrifuged at 350 g. The supernatant was aspirated and the cell pellet resuspended in said serum-free proliferation medium. The cells were plated in said serum-free proliferation medium onto a Matrigel Matrix (356230 from Corning)-coated cell culture vessel at a density of 37500 cells/cm². Differentiation was induced by changing the serum-free proliferation medium to the serum-free differentiation media as indicated in Example 2 (with DMEM/F12 1:1 as basal) 24 h later.

### Results

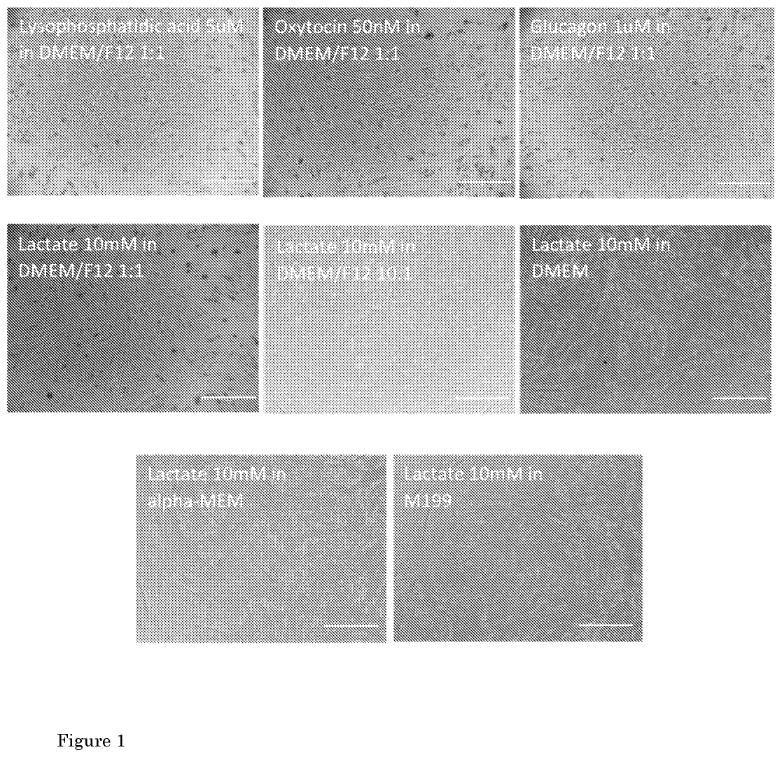

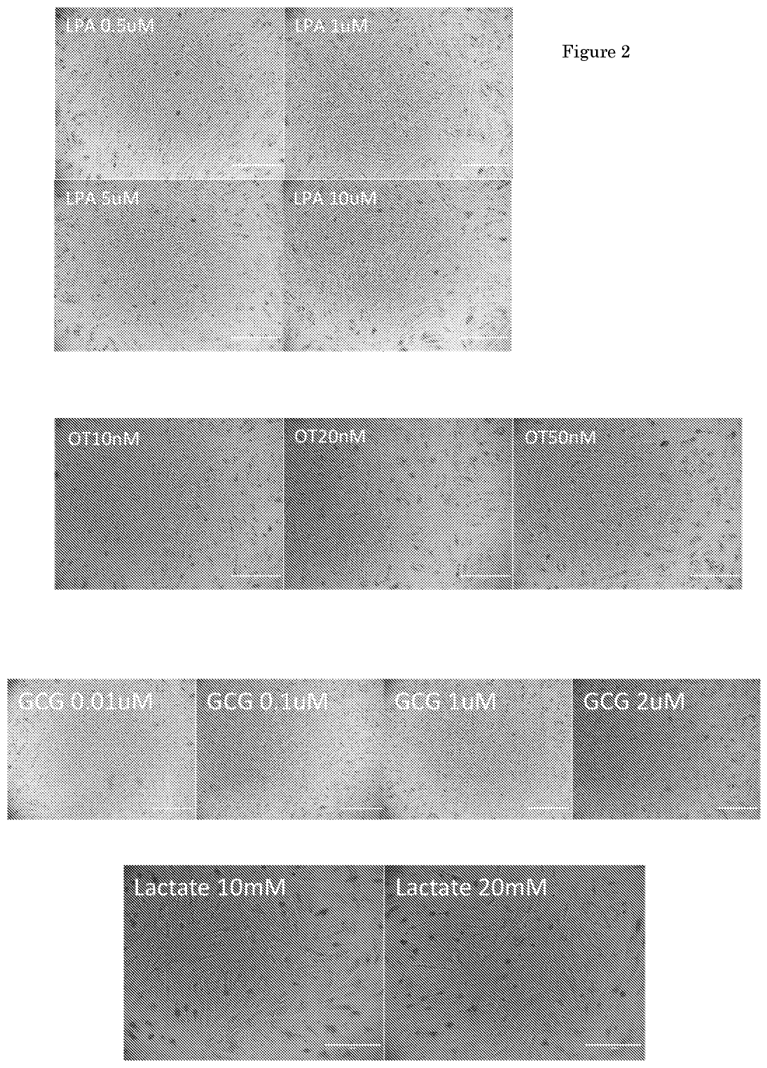

It was observed that induction of serum-free myogenic differentiation could be achieved in cells previously proliferated in serum-free proliferation medium by incorporating agonists of the identified overexpressed receptors or lactate as myogenic differentiation inducers into a serum-free differentiation medium having different basal formulations, as indicated (FIG. 1). It was further established that a range of concentrations of said differentiation inducers can successfully be employed to induce said myogenic differentiation (FIG. 2).

### Example 4. Upregulation of Notch Signaling Receptors During Myogenic Differentiation

As an add-on to Example 1, it was discovered that notch signaling receptors NOTCH2 and NOTCH3 are upregulated in a subset of satellite cells during myogenic differentiation (Table 2).

TABLE 2
Differentially expressed genes in the Notch pathway

| Ratio | | | | |
|---|---|---|---|---|
| Day 0/Day 1 | Upregulation | | | Pathway |
| (log2) | (Ratio) | Gene | Name | Inhibitor |
| −0.906 | 1.87 | NOTCH2 | notch receptor 2 | DAPT |
| −4.156 | 17.8 | NOTCH3 | notch receptor 3 | DAPT |

### Example 5. Myogenic Differentiation of Progenitor Cells with or without Notch Pathway Inhibition

### Materials and Methods

Myogenic progenitor cells as disclosed in Example 2 were plated in Matrigel-coated (1:200 in PBS) plates at a seeding density of 40 k cells/cm\hat{ }2 and cultured for 24 h in a serum-free proliferation medium as disclosed in Example 2. Myogenic differentiation was induced by adding an exemplary serum-free differentiation medium (Table 3) in the presence and absence of DAPT (gamma-Secretase inhibitor #ab120633, abcam) at 0 μM (absence of DAPT; control medium), 1 μM, 5 μM and 10 μM. Cells were imaged with brightfield microscopy and the differentiation phenotypes were compared.

TABLE 3
Exemplary serum-free differentiation medium of the invention

| DMEM (Gibco catalog number 22320- | | |
|---|---|---|
| 022) + pyruvate, glutamax, glucose | | |
| ITSE (insulin-transferrin-sodium | 2% | |
| selenite-ethanolamine) | | |
| Sodium Bicarbonate | 6.5 | mM |
| MEM AA solution 50x (Gibco catalog | 0.5% | |
| number 11130051) | | |
| Soy Hydrolysates (Sigma Aldrich - | 1% | |
| 58903C) | | |
| PSA (Penicillin Streptomycin | 1% | |
| Amphotericin) | | |
| Lactate (inducer) | 10 | mM |
| Vitamin C | 40 | μM |
| Albumin | 0.5 | mg/ml |
| EGF | 10 | ng/ml |

### Results

It was observed that Notch signaling inhibits myogenic differentiation in a subpopulation of bovine satellite cells, which can be improved by the addition of DAPT, a gamma secretase inhibitor. FIG. 3 shows improved myogenic differentiation of bovine muscle progenitor cells into myocytes in a serum-free myogenic differentiation medium with the addition of DAPT in a range of concentration (1 uM, 5 uM and 10 uM) as compared to the control medium which does not include DAPT. It was observed that the addition of the γ-secretase inhibitor DAPT during myogenic differentiation increases the number of cells that fuse into myotubes. DAPT was shown to improve differentiation at various concentrations ranging between 1 and 10 uM.

1. A method for differentiating a muscle progenitor cell, comprising the step of:
culturing a muscle progenitor cell in a serum-free medium for differentiating a muscle progenitor cell, wherein said serum-free medium comprises
at least one differentiation inducer selected from the group consisting of a lysophosphatidic acid receptor 1 (LPAR1) agonist, a lysophosphatidic acid receptor 3 (LPAR3) agonist, an oxytocin receptor (OXTR) agonist, a glucagon receptor (GCGR) agonist, a lactate and a Notch signaling pathway inhibitor.

2. The method according to claim 1, wherein said serum-free medium comprises (i) at least one differentiation inducer selected from the group consisting of a lysophosphatidic acid receptor 1 (LPAR1) agonist, a lysophosphatidic acid receptor 3 (LPAR3) agonist, an oxytocin receptor (OXTR) agonist, a glucagon receptor (GCGR) agonist and a lactate, and (ii) a Notch signaling pathway inhibitor.

3. The method according to claim 1, wherein said lysophosphatidic acid receptor 1 (LPAR1) agonist and/or said lysophosphatidic acid receptor 3 (LPAR3) agonist is a lysophosphatidic acid.

4. The method according to claim 1, wherein said oxytocin receptor (OXTR) agonist is oxytocin.

5. The method according to claim 1, wherein said glucagon receptor (GCGR) agonist is glucagon.

6. The method according to claim 1, wherein said Notch signaling pathway inhibitor is a gamma-secretase inhibitor.

7. The method according to claim 1, wherein said Notch signaling pathway inhibitor is a compound selected from the group consisting of DAPT, E2012, L685458, R04929097 and LY-411575.

8. The method according to claim 1, wherein said method is a method for proliferating a muscle progenitor cell followed by differentiating proliferated muscle progenitor cells, wherein said method further comprises, prior to differentiating said muscle progenitor cell, a step of:
culturing a muscle progenitor cell in a serum-free medium for proliferating muscle progenitor cells, to thereby provide proliferated muscle progenitor cells.

9. The method according to claim 1, wherein said method for differentiating and/or said method for proliferating of a muscle progenitor cell followed by differentiating proliferated muscle progenitor cells, is an (entirely) serum-free method.

10. The method according to claim 1, wherein said muscle progenitor cell is a bovine muscle progenitor cell, preferably a bovine (myo)satellite cell.

11. The method according to claim 1, wherein said culturing of said muscle progenitor cell in said serum-free medium for differentiating is performed under conditions that allow for differentiation of said muscle progenitor cell into a myocyte, myotube and/or myofiber.

12. The method according to claim 11, further comprising the step of:
incorporating said myocyte, myotube and/or myofiber into a meat product for human consumption, optionally in combination with adipocytes.

13. The method according to claim 1, wherein the serum-free medium for differentiating further comprises:
(i) an epidermal growth factor (EGF) or a replacement thereof;
(ii) an albumin or a replacement thereof;
(iii) a source of glucose and/or a source of glutamine;
(iv) a source of iron or an iron transporter;
(v) ascorbic acid or a derivative thereof;
(vi) sodium selenite;
(v) ethanolamine;
(vi) insulin; or
(vii) sodium bicarbonate.

14-21. (canceled)

22. The method according to claim 1, wherein the serum-free medium for differentiating comprises:
at least one differentiation inducer selected from the group consisting of a lysophosphatidic acid receptor 1 (LPAR1) agonist, a lysophosphatidic acid receptor 3 (LPAR3) agonist, an oxytocin receptor (OXTR) agonist, a glucagon receptor (GCGR) agonist and a lactate;
an epidermal growth factor (EGF) or a replacement thereof;
an albumin or a replacement thereof;
a source of glucose and a source of glutamine;
a source of iron or an iron transporter;
ascorbic acid or a derivative thereof;
sodium selenite;
ethanolamine;
insulin; and
sodium bicarbonate;
and optionally a Notch signaling pathway inhibitor.

23. The method according to claim 1, wherein all components in said serum-free medium for differentiating and/or proliferating are animal-free.

24. A serum-free medium for differentiating a muscle progenitor cell, wherein said medium is as defined in claim 1.

25. A composition comprising a serum-free medium for differentiating as defined in claim 24 and a muscle progenitor cell and/or a partially or terminally differentiated cell such as a myocyte, myotube and/or myofiber.

26. The serum-free medium according to claim 24, wherein said muscle progenitor cell is a bovine muscle progenitor cell.

27. A culture of myocytes, myotubes and/or myofibers obtainable by a method according to claim 1.

28. A meat product, comprising myocytes, myotubes and/or myofibers obtainable by a method according to claim 1.

29. The culture according to claim 27, wherein said culture is devoid of serum components such as fetal bovine serum components.

30. The composition according to claim 25, wherein said muscle progenitor cell is a bovine muscle progenitor cell.

31. The meat product according to claim 28, wherein said culture or said meat product is devoid of serum components such as fetal bovine serum components.

32. The meat product of claim 28 further comprising adipocytes

33. The meat product of claim 32 wherein the adipocytes are bovine adipocytes.
